(19) United States
(12) Patent Application Publication
(10) Pub. No.: US 2012/0252723 A1
(43) Pub. Date: Oct. 4, 2012

(54) Regulating the Amino Acid Pool Used for the Acute-Phase Protein Synthesis

(75) Inventors: Klaske van Norren, Renkum (NL); Johannes Adrianus Cornelis Peters, Den Haag (NL); Mirian Lansink, Utrecht (NL)

(73) Assignee: N.V. Nutricia, Zoetermeer (NL)

(21) Appl. No.: 13/391,398

(22) PCT Filed: Aug. 23, 2010
(86) PCT No.: PCT/NL10/50526

§ 371 Date: Apr. 12, 2012

(30) Foreign Application Priority Data
Aug. 21, 2009 (NL) PCT/NL2009/050506

(51) Int. Cl. A61K 31/198; A61K 31/405; A61K 38/02; A61P 29/00; A61P 31/00; A61P 35/00; A61P 31/18; A61P 37/00; A61P 11/00; A61P 13/12; A61P 9/04; A61P 3/08; A61P 1/16; A61P 1/18; A61P 19/02; A61P 1/00; A61P 37/08; A61P 3/02; A61K 31/202

(52) U.S. Cl. 514/5.5; 514/562; 514/560; 514/419

(57) ABSTRACT

The present invention is directed at a combination of (i) serine, (ii) cysteine, (iii) arginine and (iv) at least one branched amino acid, for use in the therapeutic or prophylactic treatment of inflammation or infection, wherein the combination is to be administered enterally and at a combination of (i) serine, (ii) cysteine, (iii) arginine and (iv) at least one branched amino acid, for use in the therapeutic or prophylactic treatment of an imbalance in the metabolic use of amino acid resources from the body or from nutrition in a subject having an inflammation or an infection, wherein the combination is to be administered enterally.

The invention is directed to the use of a combination of specific amino acids for the therapeutic or prophylactic treatment of an imbalance in the metabolic use of amino acid resources from the body or from nutrition for acute-phase protein synthesis. The invention is further directed to a specific nutritional composition.

Acute-phase proteins are a class of proteins whose plasma concentrations increase (positive acute-phase proteins) or decrease (negative acute-phase proteins) in response to infection or inflammation. This response is called the acute-phase reaction (also called acute-phase response).

Acute-phase proteins are synthesised almost exclusively by the liver. Post-traumatic changes in serum protein composition have been described as a re-prioritisation of liver protein synthesis, where the available synthetic capacity and amino acid resources are shifted from constitutive protein production to increased acute-phase protein production.

Acute-phase proteins serve important functions in restoring homeostasis after infection or inflammation. Functions that involve acute phase proteins include haemostatic functions (e.g. fibrinogen as the acute-phase protein), microbiocidal and phagocytic functions (e.g. acute-phase proteins such as complement components, C-reactive protein), anti-thrombotic properties (e.g. α1-acid glycoprotein as the acute-phase protein), and anti-proteolytic actions which are important to contain protease activity at sites of inflammation (e.g. acute-phase proteins such as α2-macroglobulin, α1-antitrypsin, and α1-antichymotrypsin).

The concentration of albumin, a continually produced acute-phase protein, usually decreases with increasing inflammatory state, because it either disappears from the veins into the intercellular space or is used in higher amounts by the body. Therefore, it has been referred to as a negative acute-phase protein. This reduction in albumin concentration coincides with an increase in inducible acute-phase proteins like C-reactive protein (CRP)—expressed especially in relation to inflammation—and with a reduction in muscle protein mass and with general malnutrition. Therefore the ratio of albumin to CRP can be used to determine the balance or imbalance between acute-phase protein synthesis and general protein metabolism.

Weight loss and impaired nutritional status are associated with increased complications following infection or any other form of metabolic stress in which an acute-phase response is involved. Protein (energy) malnutrition (PEM) may affect the acute-phase response by reducing the availability of precursors (i.e. amino acids) for acute-phase protein synthesis. However, the acute-phase protein response still persists during malnutrition. In these circumstances, the acute-phase protein response is associated with abnormally high loss of body nitrogen (>150 mg/kg body weight per day)¹. The synthesis of acute-phase proteins at the cost of skeletal muscle proteins may thus be detrimental to the body's nitrogen economy.

It has been known for decades that amino acid mixtures may be used to overcome an imbalance in plasma amino acid composition or for prophylactic or therapeutic treatment of other disorders.

For example, in WO 83/03 969 a method is contemplated for promoting growth of lean body mass by administering a parenteral amino acid mixture consisting of essential and non-essential amino acids.

In EP 147 682 A1, it was proposed to treat patients with a parenteral solution comprising a mixture of at least 14 amino acids, to prevent body-protein catabolism in convalescent patients in general.

WO 2008/015374 relates to a composition comprising free amino acids as the sole source of proteinaceous matter, long chain polyunsaturated fatty acids, carbohydrates, and bifido bacteria for treatment of a variety of disorders.

The abstract of CN 101 049 500A describes an amino acid composition comprising about 20 amino acids for preparing an injectable medicine for nutrition therapy or nutrition support (i.e. a composition for parenteral administration), amongst others for curing and improving amino acid maladjustments and hypoproteinaemia. This document is silent on which role specific amino acids play, alone or in combination. This document is also silent on acute-phase protein synthesis.

The abstract of JP 01 301 619 A describes an amino acid composition comprising about 20 amino acids for administration to cancer patients. The ratio essential to non-essential amino acids is at least 3.0. The purpose of administration is to inhibit the preferential intake of plasma proteins by cancer cells. This document is silent on which role specific amino acids play, alone or in combination. This document is also silent on acute-phase protein synthesis.

EP 655 244 A1 is directed to a composition comprising amino acids suitable for use in the treatment of tissue-damage by metabolic dysfunctioning. The dysfunctioning may be the result of an infection or an inflammatory reaction. However, this document is silent on specific amino acids as active ingredients used in combination for the treatment of an imbalance in the metabolic use of amino acid resources for acute phase protein synthesis.

U.S. Pat. No. 4,780,475 disclose a method of preventing catabolism and increasing protein synthesis by adding a mix of essential and non-essential amino acids. The mix typically contains (almost) all proteinogenic amino acids, and 40-50% of the amino acids are branched amino acids. It is not mentioned to provide the amino acids or a part thereof as (intact) protein.

EP 747 395 A1 relates to a composition comprising 18 amino acids for treating a renal failure.

EP 150 053 A2 describes a preparation comprising various amino acids and D-xylitol for the treatment of patients suffering severe stress or injury or significant renal disease to reduce nitrogen wasting and its accelerated gluconeogenesis. The preparation is for infusion (i.e. parenteral administration). The pharmacological effect is attributed to the composition being a hypocaloric amino acid and D-xylitol solution, rather than to a specific combination of amino acids.

WO 91/09524 relates to the use of arginine or ornithine for treating an impaired immune response. Optionally, the composition further comprises various other amino acids. Little or no details are given about a specific purpose for each of these other amino acids specifically.

The abstract of CN 101 332 209 A describes an injection comprising about 17 amino acids for treating hepatic encephalopathy. This document is silent on which role specific amino acids play, alone or in combination.

The abstract of JP 02 068 514 A describes a composition comprising about 11 amino acids which are said to show improvement of hypoproteinemia and hypoalbuminemia and inhibition of proliferation of tumor in Yoshida sarcoma-bearing rats. The most abundant amino acid is arginine. This document is silent on which role specific amino acids play, alone or in combination.

Thus, although (complex) mixtures comprising amino acids, optionally in combination with other compounds has been proposed for use in the treatment of various disorders, none of the above publications discloses specifically the use of the amino acids serine, cysteine, arginine and a branched amino acid as active components in combination in the treatment of an imbalance in the metabolic use of amino acid resources, in particular for acute-phase protein synthesis. Further, it is apparent that the above cited prior art does not specifically disclose the use of said amino acids as active components in combination for use in the treatment of inflammation or infection, wherein the combination is to be administered enterally.

When the amino acid composition of a typical acute-phase protein response is compared to the amino acid composition of skeletal muscle, the first limiting amino acids would be the aromatic amino acids. However, in contrast to what is to be expected, phenylalanine and tryptophan plasma concentrations seem to increase instead of decrease during infection'. Hence, just a simple supplementation of the theoretically limiting amino acids is not likely to overcome the catabolic status of the subject during an acute-phase protein response. In addition, although the acute-phase protein response persists in the fasted state, protein deficiency can alter the pattern and magnitude of the acute-phase responses in circulating protein concentrations to an extent that is dependent on the severity of protein deficiency²⁻⁴.

It is an object of the present invention to provide a novel and inventive means to prophylactically or therapeutically treat inflammation or infection, in particular to therapeutically or prophylactically treat an imbalance in the metabolic use of amino acid resources from the body or from nutrition for acute-phase protein synthesis in a subject having an inflammation or an infection.

It has been found that this object is realised by providing a combination of specific amino acids for such purpose. Accordingly, the present invention relates to a combination of (i) serine, (ii) cysteine, (iii) arginine and (iv) at least one branched amino acid, for use in the therapeutic or prophylactic treatment of inflammation or infection, wherein the combination is to be administered enterally.

In a preferred embodiment, the present invention relates to a combination of (i) serine, (ii) cysteine, (iii) arginine and (iv) at least one branched amino acid (i.e. leucine, isoleucine, valine) for use in the therapeutic or prophylactic treatment of an imbalance in the metabolic use of amino acid resources from the body or from nutrition, in a subject having an inflammation or an infection, wherein the combination is for enteral administration, in particular for oral administration. The imbalance preferably is an imbalance in the metabolic use of amino acids for acute-phase protein synthesis.

Further, the invention relates to a specific nutritional composition, namely a nutritional composition, in particular a nutritional composition suitable for oral or other enteral administration, comprising 4-9 weight % serine, 0.7-8 weight % cysteine, 2-4 weight % arginine, 8.0-20 weight % leucine, 3-8.0 weight % isoleucine, 3-8.0 weight % valine, 2.5-8.0 weight % phenylalanine, 3-7 weight % threonine, 1.0-3 weight % trypthophan, 1.0-3.0 weight % methionine, 7.5-12 weight % lysine, and 0-10 weight % glycine, all based on total weight of proteinaceous matter. Such composition may be used therapeutically to treat an inflammation or infection or prophylactically in order to avoid an inflammation or infection. Said nutritional composition is in particular suitable for use in the therapeutic or prophylactic treatment of an imbalance in the use of amino acid resources from the body or from nutrition for acute-phase protein synthesis, in a subject suffering from inflammation or infection. Advantageously, said nutritional composition further comprises histidine, in particular in a concentration of up to 7 weight %.

When herein after “cysteine” is mentioned, this term is meant to include cysteine equivalents. Cysteine equivalents are cysteine derivatives which comprise one or more cysteine units which compounds can be converted in the body to provide cysteine. In particular, cysteine equivalents are cystine (a dimer of cysteine), selenocysteine, N-acetyl cysteine, diacetyl cysteine and gluthatione.

The term “or” as used herein means “and/or” unless specified other wise.

The term “a” or “an” as used herein means “at least one” unless specified other wise.

When referring to a ‘noun’ (e.g. a compound, an additive etc.) in singular, the plural is meant to be included, unless specified otherwise.

The term ‘enteral’ is used herein for any form of administration wherein the product is allowed to enter the gut. Enteral administration includes administration in mouth, eosophagus, stomach or duodenum or ileum, In the gut the product is typically digested to a form that is absorbed (into the blood system of the subject to which the combination or product has been administered).

### DESCRIPTION OF THE FIGURES

FIG. 1. Effects of IL-6 on fibrinogen and albumin expression in HepG2 cells (n=6); IL-6 significantly induces fibrinogen excretion from HepG2 cells (p<0.001), but it has no effect on albumin excretion.

FIG. 2. Fibrinogen synthesis by HepG2 cells under influence of cytokines, dexamethasone and insulin. Each data point represents mean±STD of six experiments. IL-6 at 10 ng/ml; IL-1β at 10 ng/ml; TNFα at 10 ng/ml; dexamethasone at 1 μM and insulin at 1 μM. dex=dexamethasone (n=6; *P<0.05; **P<0.01; ***P<0.001 compared with control; #P<0.05; ##P<0.01; ###P<0.001 compared with IL-6).

FIG. 3. Fibrinogen (3A) and albumin (3B) synthesis in control and media with only essential amino acids (EAA) (n=6). The excretion of both proteins in the media decreases significantly (fibrinogen p<0.01; albumin p<0.001) when only essential amino acids are present in the media.

FIG. 4. Fibrinogen secretion of HepG2 cells in media in which one amino acid was omitted. Fibrinogen secretion was significantly inhibited when either cysteine, arginine or serine were omitted.

FIG. 5. Albumin secretion of HepG2 cells in media in which one amino acid was omitted. Albumin secretion was significantly inhibited when either cysteine, arginine or serine were omitted.

FIG. 6. Fibrinogen and albumin synthesis in control and media with only essential amino acids (EAA) or EAA with arginine, cysteine and serine. The excretion of both proteins in the media decreases significantly (fibrinogen p<0.01; albumin p<0.001) when only essential amino acids are present in the media.

FIG. 7. Albumin and fibrinogen secretion of HepG2 cells as function of total amino acid concentration in the media (n=6). Both fibrinogen and albumin synthesis increase with increasing amino acid concentrations in the media. This increase is linearly from 0 to 3000 μM, but increases dramatically between 3000 and 6000 μM.

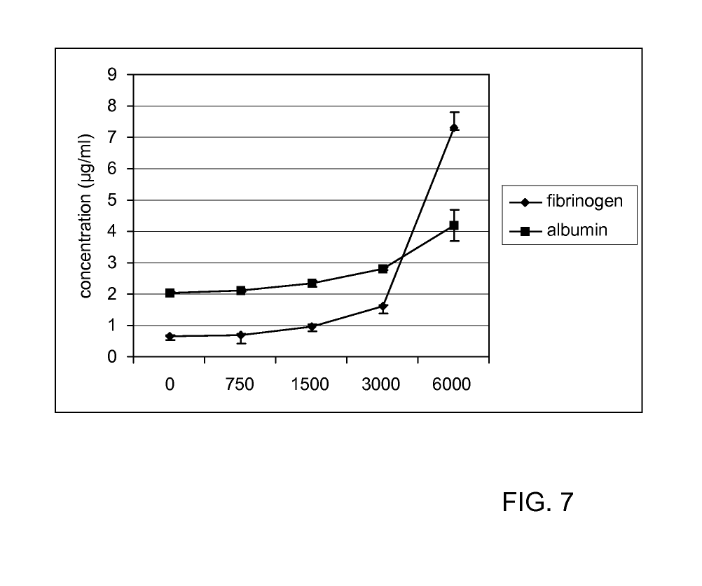

### DETAILED DESCRIPTION OF THE INVENTION

The combination for use in accordance with the invention may be or be part of a nutritional composition, nutraceutical composition or a pharmaceutical composition. The combination is for enteral administration, in particular for oral administration. The inventors contemplate that such administration is more efficient than, e.g., parenteral administration, in that a larger fraction of the administered amino acids for use according to the invention is used by the subject, especially for acute-phase protein synthesis, compared to parenteral administration. Without being bound by theory, it is believed that enteral administration of the amino acids according to the invention has a more topical character than parenteral administration in that a larger fraction is effectively delivered to the liver, where acute-phase proteins are synthesised. Further, a composition for enteral composition may comprise one or more components, in particular one or more polypeptides, which may comprise one or more allergenic epitopes, which make the composition unsuitable for parenteral administration, as this might result in an allergic or rejection reaction.

Enteral administration may be orally or by tube feeding. The administration may be carried out based on a manner known per se for a specific type of a nutritional composition, a nutraceutical composition or a pharmaceutical, e.g. a drink.

The subject to be treated may in particular be a human, although other mammals or other vertebrates may be treated in accordance with the invention. When herein after referred to clinical values, dosages and the like, these are in particular considered to relate to humans, especially adults, unless specified otherwise.

The subject to be treated may in principle be any subject in particular any subject having an inflammation or infection, having an imbalance in the metabolic use of amino acid resources from the body or from nutrition for acute-phase protein synthesis, or a subject belonging to a risk group for developing such imbalance. The blood plasma of a subject having inflammation (or infection) may in particular have an abnormally high IL-6 induced acute-phase protein level, more in particular the concentration of one or more of the IL-6 induced acute-phase proteins may be at least 25% above normal value.

The subject to be treated may in particular be a cancer patient, a subject infected with a virus, a bacterium or an other pathogenic organism, a subject having an infection, a subject having chronic obstructive pulmonary disease COPD, a subject with renal failure, a subject with heart failure, or a subject with insulin resistance. Such subjects are in particular considered to belong to a risk group for developing an imbalance in acute-phase protein synthesis, if not already having such imbalance. Of the subjects having an infection AIDS patients and HIV-infected subjects may in particular be treated with a combination or nutritional composition in accordance with the invention.

Further, the combination or nutritional composition may in particular be used in a treatment of a subject having rheumatoid arthritis or osteoarthritis, a subject having pancreatitis, a subject having hepatitis, a subject having inflammatory bowel disease, a subject having Crohn's disease (wherein the treatment is in an acute episode of said disease), a subject having an organ graft (in particular such subject suffering from rejection reactions), a subject recovering from lesions applied to a body during surgery, a critically-ill subject, a terminally ill subject, a subject suffering from an allergy (in particular a subject suffering from a severe form of allergy, i.e. a subject who is at risk of getting an anaphylactic shock, as a result of the allergy. In a further embodiment, the combination of composition is used for the treatment of an intoxicated individual.

The imbalance in acute-phase protein synthesis may be an imbalance in the recruitment of amino acids used for the synthesis of a negative acute-phase protein, such as albumin, or a positive acute-phase protein. In particular, the imbalance in acute-phase protein synthesis may be an imbalance in the recruitment of amino acids used for the synthesis of one or more proteins selected from the group of haptoglobulin (e.g. CRP), serum amyloid protein, mannose binding protein, ferritin, ceruloplasmin, serpin, tyransthyretin, transferrin, fibrinogen, and transcortin

In a specific embodiment, the acute phase protein is selected from CRP and fibrinogen.

In a specific embodiment, the combination or composition is used for the treatment of a subject, wherein the concentration of an acute phase protein in blood of the subject to be treated is more than 25% above a maximum normal physiological value in healthy individuals or more than 25% below a minimum normal physiological value in healthy individuals. The skilled person will be able to determine what normal physiological values in healthy condition are, depending on factors such as species, gender, age and the like, and common general knowledge. Normal values for various acute-phase proteins can e.g. be found in Clinical Chemistry Handbooks (e.g. “Laboratorium Informatie Gids, Diagnostisch Centrum SSDZ Delft, Reinier de Graafweg 7 2625 AD Delft. ISBN: 90-803444-1-9).

In particular in humans, normal values for fibrinogen in plasma are 2-4 g/l, normal values for haptoglobulin in serum are 0.3-2 g/l. For ferritin, normal values in serum are 10-150 μg/1 for children, 25-250 mg/l for adult men and 20-150 μg/l for adult women. In particular, in humans normal values for ceruplasmine are 0.2-0.6 g/l (in serum), whereas normal values for transferinne are 2-3.6 g/l (in serum).

In particular in humans, normal serum albumin concentrations are generally in the range of 37-53 g/l. An imbalance in albumin synthesis may in particular result in too low serum albumin levels (<37 g/l) Thus, in a specific embodiment, the treatment is directed at maintaining or restoring the normal serum albumin concentration.

In a specific embodiment, the imbalance comprises an imbalance in recruitment of amino acids for interleukin 6 (IL-6) inducible acute-phase proteins, like fibrinogen, from muscle. An imbalance in fibrinogen synthesis may in particular result in a fibrinogen concentration in venous blood outside the range of 1.0-3.0 g/1.

In a specific embodiment, the imbalance to be treated is an imbalance in the CRP synthesis, in particular an increased CRP synthesis. An imbalance in CRP synthesis can be determined by measuring serum CRP levels. Too high levels of serum CRP are defined as more than 10 mg/l (0.01 g/l), if the serum albumin concentration is normal. If the albumin concentration is below normal value (<37 g/l), an imbalance in CRP synthesis can be determined by determining the weight to weight ratio of serum CRP to serum albumin. If the ratio CRP in mg/l to serum albumin in g/l, is higher than 0.27, an imbalance in CRP exists. The CRP-to-albumin ratio is a particularly suitable parameter to determine the existence of an imbalance in the metabolic use of amino acid resources from the body or from nutrition for acute-phase protein synthesis. An imbalance in CRP synthesis may already be noticeable, whilst the fibrinogen and/or albumin levels are still within a normal range. Thus, the use of CRP as an indicator for an imbalance in the metabolic use of amino acid resources is in particular also suitable for determining the presence of a relatively mild imbalance.

The invention is therefore in particular considered advantageous in that it not only provides for a treatment of an imbalance in the use of amino acid resources from the body versus nutrition for the synthesis of a negative acute-phase protein (albumin, pre-albumin), but also for a treatment of an imbalance in the synthesis of a positive, IL-6 inducible acute-phase protein (CRP, fibrinogen).

The invention is considered in particular advantageous, in that administration of a combination or nutritional composition according to the invention to a subject experiencing an inflammatory response, reduces the break-down of muscle protein for the acute-phase protein response. This results in better maintenance of muscle or lean body mass.

It is contemplated that a combination or nutritional composition according to the invention may be used for a treatment of an imbalance in the use of amino acid resources from the body or from nutrition for one or more other positive phase acute-phase proteins, such as complement factors, C-reactive protein, α1-antitrypsin and/or α1-antichymotrypsin.

A combination or nutritional composition for treatment of an imbalance in the metabolic use of amino acid resources may in particular be useful for restoring acute-phase protein homeostasis after infection or inflammation.

The inventors have further come to the conclusion that the invention is in particular advantageous in that it can help to avoid or at least reduce muscle-protein catabolism associated with an imbalance in acute-phase protein synthesis. To this purpose, an enteral administration is considered to be particular beneficial, compared to, e.g., parenteral administration. Thus, in accordance with the invention, a combination for use in accordance with the invention may in particular be used for shifting the amino acid source for acute-phase protein synthesis from body protein to administered amino acid or amino acids in the combination or composition of the invention.

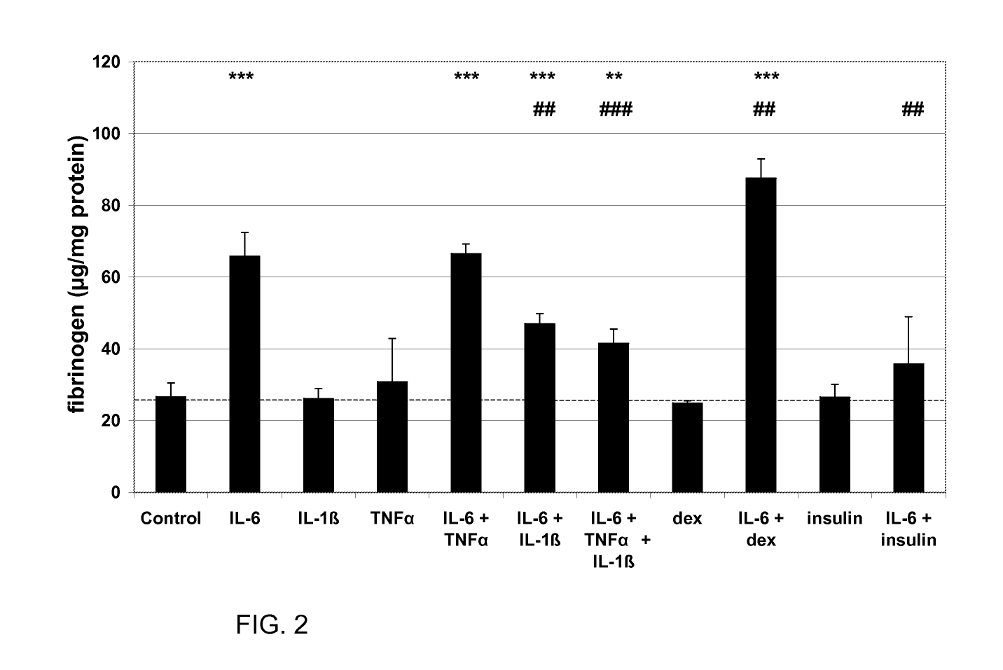

In a specific embodiment, the combination for use according to the invention or the nutritional composition according to the invention is used for a medical treatment (or is for use in a medical treatment) wherein the subject to be treated has insulin resistance (IR) or diabetes. IR or diabetes may be accompanied by an increased acute-phase protein response. It has been found that insulin has an inhibitory effect on the IL-6 induced acute-phase protein synthesis. This is illustrated in the Examples and the Figures, see Example 2 and FIG. 2. Thus, the inventors conclude that a relative lack of insulin may contribute to muscle protein catabolism. In particular, if the combination of the invention is used for treatment of a subject having IR or diabetes, the combination preferably comprises leucine, which may have a stimulating effect on insulin secretion, and/or the combination may be part of a nutritional composition, in particular a composition for enteral administration that has a low glycaemic index, which may in particular contribute to improving insulin sensitivity of the subject.

A combination or composition according to the invention, in particular a combination for the treatment of a subject having IR or diabetes advantageously has a glycaemic index (GI) below 55. Preferably, the GI is 0-45. In practice, the glycaemic index will usually be above zero, at least in a nutritional composition comprising a digestible carbohydrate. In particular, the GI will be at least 1, more in particular at least 5. Details on how to determine the glycaemic index of a composition are provided in the Examples, herein below.

The skilled person will be able to formulate a composition with a relatively low glycaemic index based on the information disclosed herein and common general knowledge. In particular, by increasing the percentage of carbohydrate that is digested more slowly than glucose or by increasing carbohydrates that provide less glucose moieties per weight than glucose, the glycaemic index of a composition (under otherwise the same condition) is decreased. Preferred examples of carbohydrates which are digested more slowly than glucose are isomaltulose, fructose, galactose, lactose and trehalose. Furthermore, the addition of fat and fibre can slow down gastric emptying. Moreover, fibres can form a physical barrier in the intestine, reducing absorption rate. Amino acids from proteins can increase insulin release (especially leucine), and thereby increase glucose uptake by the cells. All these mechanisms can contribute to a reduction in glycaemic index.

Administration of a combination or nutritional composition having a GI below 55, preferably of 45 or less is, amongst others, considered advantageous in a subject having a chronic inflammation as a result of a prolonged too high glucose level in the blood, such as a diabetic subject. A low GI combination according to the invention may also reduce the likelihood of developing such inflammation.

Further, the inventors contemplate that a combination or nutritional composition (for use) according to the invention having a GI below 55, preferably of 45 or less, is advantageous in that it can be administered orally without giving rise to a (substantial) risk of causing or contributing to hypoglycaemia, whilst it is helpful in reducing (the risk of an infectious complication. Examples of infectious complications that may be reduced or of which the risk may be reduced in particular include postoperative infectious complications, prevention of sepsis, increased sensitivity to develop infections to the airways, pneumonia, bronchitis, ulcers and delayed wound healing and an increased severity of the infections. It is noted that intravenous injections with amino acid compositions have been associated with decreases in infectious complications. However, such treatment has a considerable disadvantage in that it may cause or contribute to the occurrence of hypoglycaemia, which may result in coma or death.

A combination or nutritional composition according to the invention, in particular such combination or composition for the treatment of a subject having IR or diabetes, may further comprise an anti-inflammatory component, such as an ω-3 polyunsaturated fatty acid.

In a specific embodiment, the combination or composition (for use) according to the invention, is used in a medical treatment wherein the subject to be treated has cachexia, wherein the treatment is directed at avoiding or reducing tissue catabolism in the subject, in particular muscle catabolism.

Such a positive effect on muscle-catabolism is unexpected, in view of prior art publications. Weight loss (at least partially due to muscle protein loss) and impaired nutritional status are associated with increased complications following infection or any other form of metabolic stress in which an acute-phase response is involved. Severe malnutrition may affect the acute-phase response by reducing the availability of precursors (amino acids) for acute-phase protein synthesis. However, the acute-phase protein response still persists during malnutrition.^{5,6} Under these circumstances the acute-phase protein response is associated with a loss of body nitrogen (>150 mg/kg body weight per day). The synthesis of acute-phase proteins at the cost of skeletal muscle proteins may thus be detrimental to the body's nitrogen economy. Theoretically, an imbalance could be overcome by adding the limiting amino acids to the diet of the subject. When the amino acid composition of a typical acute-phase protein response is compared to the amino acid composition of skeletal muscle, the first limiting amino acids would be the aromatic amino acids⁷. However, in contrast to what is to be expected, it was concluded that phenylalanine and tryptophan plasma concentrations seem to increase instead of decrease during infection. Hence, it can be concluded that just a simple supplementation of the theoretically limiting amino acids is not likely to overcome the catabolic status of the subject during an acute-phase protein response. The inventors contemplate that in particular a combination or nutritional composition further comprising one or more omega-3 fatty acids, more in particular one or more omega-3 fatty acids selected from the group of EPA, DHA and DPA is beneficial with respect to the treatment in accordance with the invention of a subject having cachexia or with respect to reducing the risk of developing cachexia.

The term amino acid is used herein for the proteinogenic amino acids, i.e. those amino acids that are found in proteins and that are coded for in the standard genetic code. Proteinogenic amino acids include: alanine, valine, leucine, isoleucine, serine, threonine, methionine, cysteine, asparagine, glutamine, tyrosine, tryptophan, glycine, aspartic acid, glutamic acid, histidine, lysine, arginine, proline and phenylalanine.

When referred herein to an amino acid, this term includes amino-acid residues, especially amino acid residues in peptides, and—in the case of cysteine—cystine (a dimer of cysteine) selenocysteine, N-acetyl cysteine and other equivalents.

When referred herein to the term ‘proteinaceous matter’, this includes free amino acids (including its zwitter ionic state or other ionic state), amino acid salts, amino acid esters, the amino acid residues bound to conjugating molecules and peptides, including proteins. Likewise, when reference is made to a specific amino acid, e.g. serine, cysteine, arginine, a branched amino acid or another essential amino acid, this is meant to include the specific amino acid (residues) present as a salt, in a bound form, as well as the free specific amino acid.

Amino acids may in particular be provided by any source which allows the provision of the free amino acid when properly administered to the body, in particular by any source that provides the free amino acid, upon digestion of the source. An amino acid source may in particular comprise at least one of the following compounds: amino acid in the form of a free acid (including its zwitter ionic or other state); amino acid salts; peptides; proteins; conjugates of an amino acid leucine with a conjugating compound other than an amino acid, a protein, or a peptide, which conjugate is capable of being split into the free amino acid (or salt thereof), preferably in the gut or stomach or after absorption in the enterocytes or liver. In addition, cystine is a suitable source for free cysteine.

With a peptide is meant a combination of two or more amino acids, connected via one or more peptidic bonds. When incorporated in a peptide, amino acids are named amino-acid residues. Peptides include oligopeptides and polypeptides, including proteins.

With a polypeptide is meant a peptide chain comprising 14 or more amino-acid residues. With an oligopeptide is meant a peptide chain comprising 2-13 amino-acid residues.

Chiral amino acids present in a combination or composition of the invention are preferably in the L-form.

Hereinafter, unless specified otherwise, when referred to an amino acid, the amino acid in any form is meant, i.e. including not only the free amino acid or salt thereof, but also the amino acid in any bound form.

The proteinaceous matter may comprise one or more proteins, which protein(s) or part thereof may be intact or may have been modified, in particular by (partial) hydrolysis, usually to the extent that up to 20% of the protein is hydrolysed to free amino acids, preferably to the extent that up to 10% of the protein is hydrolysed to free amino acids. An advantage of (partially) hydrolysed protein is an advantageous amino-acid release behaviour, when enterally administered.

The peptide content (oligopeptide, polypeptide, protein) based on proteinaceous matter is usually at least 50 weight %, at least 60 weight %, or at least 75 weight %. The weight % of peptide, based on proteinaceous matter, is usually 99.5 weight % or less, preferably up to 94 weight % or less.

An advantage of a composition wherein the peptide content is high (≧50 weight %) is that the taste, or another organoleptic property of the composition, is usually appreciated better when consumed (orally). Further, the uptake of amino acids by the body may be more gradual.

The presence of peptides formed of 8 or more amino acid units is preferred in particular for improved taste of a product comprising the combination. Further, the presence of such peptides is considered advantageous for improved stability, in particular in case the product is a heat-treated (e.g. pasteurised or sterilised) liquid product. Preferably, at least 50 wt. % of the peptides is formed by one or more peptides having 8 or more amino acid units. More preferably, at least 50 wt. % of the proteinaceous matter is formed by one or more peptides having 8 or more amino acid units.

The proteinaceous matter may comprise one or more peptides having one or more allergenic epitopes (which peptides include proteins having an allergenic epitope), this in contrast to a preparation for parenteral administration. In general, such peptides are formed of 8 or more amino acid units (i.e. octapeptides and larger peptides).

If intact protein is present, the intact protein content is usually at least 10 weight %, based on total proteinaceous matter, in particular at least 25 weight %, more in particular 50 weight % or more. In principle, all proteinaceous matter may be intact protein. Usually, the intact protein content is 99.5% or less, in particular 95 wt. % or less, more in particular 90 wt. % or less, based on total proteinaceous matter.

The proteinaceous matter (providing the one or more amino acids for use in accordance with the invention) preferably comprises at least one protein selected from the group of whey proteins, casein, caseinate, soy proteins and wheat proteins, preferably from the group of whey proteins and casein.

With whey proteins are meant globular proteins that can be isolated from whey. In particular, globular whey proteins can be selected from beta-lactoglobulin, alpha-lactalbumin and serum albumin, including mixtures thereof. Examples of mixtures that comprise whey proteins are whey isolate and whey concentrate.

In an advantageous embodiment, the proteinaceous matter comprises whey protein. The presence of a whey protein may offer a number of advantages. Whey shows an advantageous release behaviour both in terms of release rate of the amino acids and the tendency to make the amino acids available for uptake by the body, essentially at the same time.

If present, the whey protein content may in particular be at least 10 weight % based upon the proteinaceous matter, preferably at least 15 weight % based upon the proteinaceous matter. In particular in case cystein is to be provided in accordance with the invention, it is particularly preferred that the whey protein content is at least 25 weight %, even more preferred more than 40 weight %, at least 45 weight %, e.g. about 50 weight % or more, based on total proteinaceous matter.

Usually, the whey protein fraction is 70 weight % or less, in particular 60 weight % or less, more in particular 55 weight % or less, based on proteinaceous matter. In a specific embodiment, the whey protein fraction is over 70 weight %, for instance in the range of 90-100 weight % whey protein, based on proteinaceous matter. Advantageously, part of the whey protein is (partially) hydrolysed, in particular up to 50 weight % of the whey protein may be (slightly) hydrolysed, in particular 10-50 weight %.

As the source for whey proteins preferably a whey fraction is chosen comprising less that 20 weight % of casein glycomacropeptide (GMP), more preferably less than 10 weight %, based on total whey fraction.

The beta-lactoglobulin content preferably is larger than 40 weight %, more preferably 46-80 weight %, based on total whey fraction. This is advantageous because beta-lactoglobulin has a relatively high leucine content.

When used as intact protein, the casein preferably comprises a high concentration of beta-casein, in particular more than 36 g beta-casein/100 g total casein, more in particular 38-70 g beta-casein/100 g total casein.

A suitable daily dosage of cysteine (in particular for an adult subject, e.g. weighing 70 kg, in particular an adult human)—optionally comprising cysteine equivalents, such as cystine, N-acetyl-cysteine and selenocysteine, as defined above—is usually 250 mg or more, in particular 340 mg or more. The daily dosage is usually 3 g or less, in particular 2 g or less.

A nutritional composition according to the invention usually comprises at least 0.7 weight %, in particular at least 0.80 weight % preferably at least 0.85 weight % or at least 0.90 weight % of cysteine. In particular said concentration may be up to 8 weight %, more in particular up to 3 weight %, based on total proteinaceous matter, if present. A suitable daily dosage of serine (in particular for an adult subject, e.g. weighing 70 kg, in particular an adult human), is usually 1.5 g or more, in particular 2.0 g or more. The daily dosage is usually 20 g or less, in particular 15 g or less.

A nutritional composition according to the invention usually comprises at least 4.0 weight %, in particular at least 5.0 weight % of serine, preferably 6 to 9 weight % of serine, based on total proteinaceous matter, if present.

A suitable daily dosage of arginine (in particular for an adult subject, e.g. weighing 70 kg, in particular an adult human), is usually 0.8 g or more, in particular 1.1 g or more. The daily dosage is usually 10 g or less, in particular 8 g or less.

A nutritional composition according to the invention usually, comprises at least 2.0 weight % of arginine, preferably 2.5-4 weight %, based on total proteinaceous matter, if present.

The relative amount of the sum of serine, cysteine, and arginine, based on the weight of total non-essential amino acids usually is at least 17.5 weight %, in particular 18.5 weight %, more in particular at least 20 weight %. In case one or more further amino acids are present, the relative amount of the sum of serine, cysteine, and arginine, based on the weight of total amino acids may be 60 weight % or less, in particular 55 weight % or less.

The relative amount of the sum of serine and cysteine, based on the weight of total amino acids, usually is at least 5 weight %, in particular 6 weight %, more in particular at least 7 weight %, or at least 7.5 weight %. The relative amount of the sum of serine and cysteine, based on the weight of total amino acids may be 60 weight % or less, in particular 55 weight % or less.

The relative amount of the sum of serine and cysteine, based on the weight of total non-essential amino acids usually is at least 11.5 weight %, in a particular at least 12.5 weight %, more in particular at least 13 weight %, or at least 15.5 weight %.

The relative amount of the sum of serine, cysteine, and arginine, based on the weight of total amino acids usually is at least 7 weight %, in particular at least 8.5 weight %, more in particular at least 10 weight %. The relative amount of the sum of serine, cysteine, and arginine, based on the weight of total amino acids may be 60 weight % or less, in particular 55 weight % or less.

The combination or nutritional composition further comprises at least one branched amino acid (i.e. leucine, isoleucine, valine). In a particular preferred embodiment, each of leucine, isoleucine, and valine are present.

The combination or nutritional composition usually comprises at least 3 weight % branched amino acid(s) based on total amino acid content. The total content of branched amino acid(s) may be 50 weight % or less, in particular 42 weight % or less, more in particular 35 weight % or less, based on total amino acid content.

If present, the leucine content based on total amino acids is usually in the range of 5-25 weight %, in particular in the range of 8-20 weight %, more in particular in the range of 10.2-19.4 weight %.

If present, the isoleucine content based on total amino acids usually is in the range of 3-8 weight %, in particular in the range of 4-7 weight %, more in particular in the range of 4.9-6.1 weight %.

If present, the valine content based on total amino acids, usually is in the range of 3-9 weight %, in particular in the range of 4-8 weight %, more in particular in the range of 5.6-6.6 weight %.

In a preferred embodiment, the combination or nutritional composition further comprises at least one essential amino acid, further to the branched amino acid or branched amino acids that are present. The further essential amino acids are selected from the group of phenylalanine, threonine, tryptophan, methionine and lysine. In a particular preferred embodiment at least three other essential amino acids are present in the combination or nutritional composition further to the branched amino acid or branched amino acids that are present as component (iv).

Preferably, each of the essential amino acids leucine, isoleucine, valine, phenylalanine, threonine, tryptophan, methionine and lysine are present in the combination or nutritional composition. The total relative amount of the sum of leucine, isoleucine, valine, phenylalanine, threonine, tryptophan, methionine and lysine, based on total amino acids in a combination or nutritional composition of the invention preferably is at least 40 weight %, in particular at least 45 weight %. more in particular at least 50 weight %. Usually said total relative amount is 80 weight % or less, in particular 70 weight % or less. The total relative amount of serine, cysteine and arginine, relative to the total amount of essential amino acids in the composition usually is at least 10 weight %, in particular at least 15 weight %, more in particular at least 20 weight %. The total relative amount of serine, cysteine and arginine, relative to the total amount of essential amino acids usually is 40 weight % or less, in particular 30 weight % or less. If present in the combination or nutritional composition of the invention, the phenylalanine content based on total amino acids usually is in the range of 2.5-8 weight %, in particular in the range of 3-7 weight %, more in particular in the range of 3.9-5.1 weight %.

If present in the combination or nutritional composition of the invention, the threonine content based on total amino acids usually is in the range of 2.5-8 weight %, in particular in the range of 3-7 weight %, more in particular in the range of 4.2-6.1 weight %.

If present in the combination or nutritional composition of the invention, the tryptophan content based on total amino acids usually is in the range of 0.5-4 weight %, in particular in the range of 1.0-3 weight %, more in particular in the range of 1.3-1.5 weight %.

If present in the combination or nutritional composition of the invention, the methionine content based on total amino acids usually is in the range of 1.0-6 weight %, in particular in the range of 1.5-4 weight %, more in particular in the range of 2.2-2.7 weight %.

If present in the combination or nutritional composition of the invention, the lysine content based on total amino acids usually is in the range of 4-15 weight %, in particular in the range of 5-12 weight %, more in particular in the range of 7.5-9.5 weight %.

If present in the combination or nutritional composition of the invention, the glycine content based on total amino acids usually is in the range of 0.5-10 weight %, in particular in the range of 1.5-8 weight %, more in particular in the range of 1.7-5 weight %.

If present in the combination or nutritional composition of the invention, the histidine content is usually in the range of 0.5-7 weight %, in particular in the range of 1-5 weight %, more in particular in the range of 2-3 weight %.

The relative amount of branched amino acids based on the total of leucine, isoleucine, valine, phenylalanine, threonine, tryptophan, methionine and lysine preferably is at least 45 weight %, in particular at least 52 weight % Usually said relative amount is 75 weight % or less, in particular 60 weight % or less.

A combination of amino acids according to the invention or nutritional composition according to the invention may further be combined with one or more additional components.

In a particularly preferred embodiment, the combination or composition comprises one or more anti-inflammatory components. Examples of nutritional components that have an anti-inflammatory effect are glycine and ω-3 polyunsaturated fatty acids. It is noted that glycine also forms part of the proteinacous matter, and that ω-3 polyunsaturated fatty acids also forms part of the group of lipids, which may be present.

Glycine may be present as a free amino acid or in an other form, e.g. in a peptidic form (e.g. collagen hydrolysate).

In a specific embodiment, the combination or nutritional composition of the invention is used in combination with a compound selected from the group of antibiotics, virus-inhibitors, chemotherapeutic agents and nutritional components capable of inhibiting COX II activity, e.g. curcumine.

In particular, it is considered advantageous with respect to treatment of an imbalance in acute-phase protein synthesis to include one or more ω-3 polyunsaturated fatty acids, in particular one or more ω-3 polyunsaturated fatty acids having 18-26 carbon atoms, more in particular one or more ω-3 polyunsaturated fatty acids selected from the group of eicosapentaenoic acid (EPA), docosahexaenoic acid (DHA), eicosatetraenoic acid (ESA) and docosapentaenoic acid (DPA).

The use of one or more ω-3 polyunsaturated fatty acids in a combination with the amino acids or a nutritional composition comprising one or more ω-3 polyunsaturated fatty acids is in particular considered advantageous in that such combination or composition contributes to the shifting of the body of a subject treated with a combination or composition according to the invention from using the subject's muscle tissue as a source for acute-phase protein synthesis to using the combination or nutritional composition comprising the amino acids as a source for acute-phase protein synthesis. In particular the inventors realised that the presence of one or more ω-3 polyunsaturated fatty acids is suitable to attenuate or even completely avoid increasing the synthesis rate of CRP and/or one or more other acute-phase proteins as a result of administering the combination or nutritional composition of the invention, whilst the combination (e.g. in the form of a nutritional composition) is effective in shifting from using body-muscle tissue as a source to the amino acid(s) in the combination.

In principle, any food-grade or pharma-grade lipid or mixture of lipids comprising free unsaturated fatty acid or fatty-acid derivative (including tri-, di-, and monoglycerides and phospholipids) may serve as the fatty acid source. When referred herein to fatty acids, this is meant to include derivatives thereof, such as triglycerides, diglycerides, monoglycerides and phospholipids comprising an unsaturated fatty acid residue.

In an embodiment wherein one or more ω-3 polyunsaturated fatty acids are included, the daily dosage (in particular for a human adult) usually is at least 1.4 g., in particular at least 4 g. Usually, the daily dosage is 25 g or less, in particular 22 g or less.

In a nutritional composition according to the invention, the ω-3 polyunsaturated fatty acid content, based on total lipid content, is usually at least 9 weight %, preferably at least 15 weight %, if present. In a nutritional composition or combination according to the invention, the ω-3 polyunsaturated fatty acid content, based on total proteinaceous matter, is usually at least 4 weight %, preferably at least 10 weight %, if present.

The combination or composition may further comprise one or more additional ingredients, in particular one or more ingredients selected from the group of digestible carbohydrates, indigestible carbohydrates and other dietary fibres, trace elements, minerals, vitamins, proteinaceous matter other than proteinaceous matter providing said amino acids, other lipids than the ω-3 polyunsaturated fatty acid for treatment in accordance with the invention, and other typical additives for nutritional compositions, nutraceutical compositions or pharmaceutical compositions (such as antioxidants, flavourings, stabilising agent, or—in case of a pharmaceutical: a pharmaceutically acceptable carrier). Examples of additional ingredients are, e.g., described in WO2003/041701 (N.V. Nutricia) and WO2007/073178 (N.V. Nutricia). Examples of additional ingredients of which one or more may be present in a composition for use in accordance with the invention, in particular for a nutritional composition, in particular include sodium, potassium, chloride, fluoride, iodide, phosphorous, magnesium, vitamin A, vitamin D3, vitamin E, vitamin K, vitamin B1, vitamin B2, vitamin B3, vitamin B5, vitamin B6, folic acid, vitamin B12, biotin, vitamin C, lipoic acid, zinc, iron, copper, manganese, molybdenum selenium and chromium. Such components may be present in a concentration up to the daily recommended dose per daily serving.

In particular, of the vitamins, vitamin D3 is preferred. This vitamin is considered to be advantageous for maintenance of muscle mass, especially if a subject to be treated is an elderly person (e.g. 60 years or older). In a specific embodiment, the elderly person has a chronic inflammation and/or insuline resistance.

In this respect, it is submitted that in the context of this application, an elderly person is a person of the age of 50 or more, in particular of the age of 55 or more, more in particular of the age of 60 or more, more in particular of the age of 65 or more. This rather broad definition takes into account the fact that the average age varies between different populations, on different continents, etc. Most developed world countries have accepted the chronological age of 65 years as a definition of ‘elderly’ or older person (associated with the age at which one may begin to receive pension benefits), but like many westernized concepts, this does not adapt well to e.g. the situation in Africa. At the moment, there is no United Nations (UN) standard numerical criterion, but the UN agreed cut-off is 60+ years to refer to the older population in Western world. The more traditional African definitions of an elder or ‘elderly’ person correlate with the chronological ages of 50 to 65 years, depending on the setting, the region and the country.

In particular, one or more indigestible oligosaccharides may be present, such as one or more oligosaccharides selected from the group of galactooligosaccharides (GOS) and fructooligosaccharides (FOS). With an oligosaccharide is meant a chain comprising 2-25 saccharide residues. If present, the indigestible carbohydrate content may in particular be about 5-40 g/kg, more in particular 10-25 g/kg, based on dry weight. A daily dosage (for an adult, e.g. about 70 kg) may in particular be selected in the range of 2.5-15 g/day.

In a specific embodiment, the composition is a nutritional composition. With a nutritional composition is meant a composition that comprises naturally occurring components, preferably found in the food supply, that can be sold over the counter, as supplements, functional foods or food ingredients i.e. without a physician's or veterinarian's prescription. A nutritional composition may also be a medical food, intended for the dietary management of a disease or condition for mammals under the supervision of a physician or veterinarian.

A nutritional composition according to the invention may be in the form of a liquid, e.g. a drink, in the form of a semi-liquid, e.g. a yoghurt or a custard, in the form of a gel, e.g. jelly cake or in the form of a solid, e.g. a candy bar or an ice-cream.

In an embodiment, a liquid composition is prepared from a concentrate, e.g. from a liquid (e.g. with a viscosity of less than about 80 mPa·s), a semi-liquid (e.g. with a viscosity of more than about 80 mPa·s and less than about 400 mPa·s), a gel or a solid. For such preparation, water may be used to dilute the concentrate. In particular, such preparation occurs just before administration of the composition, e.g. in an instant-fashion.

A nutritional composition usually comprises proteinaceous matter, a lipid, and a digestible carbohydrate. In an advantageous embodiment, a nutritional composition according to the invention provides all dietary requirement, and thus may serve as the only food source for a subject (i.e. a complete nutrition). Such composition typically comprises proteinaceous matter, at least providing the essential amino acids, arginine, cystein and serine; lipids, at least providing the essential fatty acids; digestible carbohydrate; fibre; minerals and vitamins. The skilled person will be able to formulate a suitable food composition to serve as a total-food based on the present disclosure and common general knowledge; e.g., the skilled person may base a formulation on ‘Foods for Special Medical Purposes’ regulations or the like. As a rule of thumb, such food provides all needed nutrients and energy within a product volume of 2 litres or less (650 g solids or less).

A specific embodiment of the invention is a nutritional composition comprising proteinaceous matter, a lipid, and a digestible carbohydrate, wherein
a) the proteinaceous matter content provides 18-50 en %, preferably 20-40 en %, more preferably 22-32 en % of the total composition;
b) the lipid content provides 10-50 en %, preferably 20-40 en %, more preferably 25-35 en % of the total composition;
c) the digestible carbohydrate content provides 20-70 en %, preferably 30-60 en %, more preferably 38-48 en % of the total composition.

The energetic value of a compound (en %) is based on the energy provided by the digestible part (in particular in a human) of the compound. In particular, the energetic value is based on the contribution of proteinaceous matter, lipids and digestible carbohydrates, using the following calculation factors: 4 kcal/g for digestible carbohydrates and proteinaceous matter and 9 kcal/g for lipids.

The total energetic value of a liquid composition in accordance with the invention may be chosen within wide limits, e.g. from 0.2-4 kcal/ml. Usually it is at least 0.3 kcal/ml, in particular at least 0.8 kcal/ml, more in particular at least 1.2 kcal/ml. Usually, it is 3.0 kcal/ml or less, in particular 2.6 kcal/ml or less, more in particular 2.4 kcal/ml or less. In a specific embodiment, the liquid composition in accordance with the invention has an energetic value in the range of 0.3-3.0 kcal/ml, preferably 0.8-2.6 kcal/ml, more preferably 1.2-2.4 kcal/ml.

In another specific embodiment, the liquid composition in accordance with the invention has an energetic value in the range of 0.2-1.0 kcal/ml, preferably 0.4-0.9 kcal/ml.

Factors that play a role in determining a desirable energetic value include the ease of achieving a higher en % proteinaceous matter on the one hand and a fast emptying of the stomach (increasing anabolic response) on the other hand.

In a specific embodiment, the composition is a nutritional composition with a low glycaemic index. In particular a composition with a glycaemic index below 55, preferably below 45. In practice, the glycaemic index will always be above zero, and usually be at least 1, in particular at least 5. Details on how to determine the glycaemic index of a composition are provided in the Examples, herein below. The skilled person will be able to formulate a composition with a relatively low glycaemic index based on the information disclosed herein and common general knowledge. In particular, by increasing the percentage of carbohydrate that is digested more slowly than glucose or by increasing carbohydrates that provide less glucose moieties per weight than glucose, the glycaemic index of a composition (under otherwise the same condition) is decreased. Preferred examples of carbohydrates which are digested more slowly than glucose are isomaltulose, fructose, galactose, lactose and trehalose. Next to that addition of fat and fibre can slow down gastric emptying. Moreover, fibres can form a physical barrier in the intestine, reducing absorption rate. Amino acids from protein can increase insulin release (especially leucine), and thereby increase glucose uptake by the cells. All these mechanisms can contribute to a reduction in GI.

The combination or composition of the invention may be administered under the supervision of a medical specialist, or may be self-administered.

The invention will now be illustrated on the basis of the following examples.

### EXAMPLES

### Example 1

### Formulation Example

Three possible examples of sip feeds are provided in Table 1 (amounts per 100 ml are provided).

TABLE 1

| | Formu- | Formu- | Formu- | Formu- |
|---|---|---|---|---|
| | lation A | lation B | lation C | lation D |
| energy (kcal) | 152.0 | 150.0 | 160.0 | 47.0 |
| protein (g) | 10.0 | 7.5 | 10.1 | 5.0 |
| of which whey (g) | 2.0 | 3.8 | 2.9 | 2.5 |
| carbohydrates (g) | 15.6 | 22.5 | 17.4 | 2.0 |
| fat (g) | 5.3 | 3.3 | 5.3 | 2.0 |
| of which unsaturated | 4.7 | 2.7 | 4.2 | 1.5 |
| of which poly unsaturated | 1.5 | 1.9 | 2.7 | 1.0 |
| of which sum | | 0.4 | 1.1 | 0.2 |
| (EPA + DHA + DPA) | | | | |
| fibre, dietary g | 1.0 | | 2.0 | 0.5 |
| G.I. of the product | | | 40 | <55 |
| a.a. content per 100 g | | | | |
| protein | | | | |
| L-Alanine | 3.5 | 4.4 | 3.3 | 3.2 |
| L-Arginine | 3.6 | 3.4 | 2.9 | 3.3 |
| L-Aspartic acid/L- | 8.0 | 9.6 | 7.3 | 7.2 |
| Asparagine | | | | |
| L-Cyst(e)ine | 0.9 | 1.5 | 0.9 | 0.8 |
| L-Glutamic acid/L- | 22.7 | 22.1 | 17.8 | 20.5 |
| Glutamine | | | | |
| Glycine | 2.0 | 2.1 | 1.7 | 1.8 |
| L-Histidine | 3.0 | 2.5 | 2.1 | 2.7 |
| L-Isoleucine | 5.4 | 6.1 | 5 | 4.9 |
| L-Leucine | 10.2 | 10.8 | 19.4 | 12.5 |
| L-Lysine | 8.8 | 9.5 | 7.5 | 7.9 |
| L-Methionine | 2.7 | 2.6 | 2.2 | 2.4 |
| L-Phenylalanine | 5.1 | 4.6 | 3.9 | 4.6 |
| L-Proline | 10.0 | 8.5 | 8.1 | 9.0 |
| L-Serine | 6.1 | 6.3 | 5.1 | 5.5 |
| L-Threonine | 4.6 | 6.1 | 4.9 | 4.2 |
| L-Tryptophan | 1.4 | 1.5 | 1.4 | 1.3 |
| L-Tyrosine | 4.9 | 4.3 | 4.1 | 4.4 |
| L-Valine | 6.5 | 6.6 | 5.6 | 5.9 |

### Example 2

### Acute-Phase Protein Synthesis in a HepG2 Model

The effects of either the amino acid concentration or the amino acid composition on the acute-phase protein response in a human hepatocellular cell line (HepG2 cell) were studied. As model components, the production of fibrinogen and albumin were studied.

### Materials and Methods

Cell culture: Human hepatoma HepG2 cells (ATCC HB-8065) were maintained in DMEM supplemented with 10% heat inactivated fetal calf serum (Life Technologies) with 2 mM glutamine and streptomycin/penicillin in Costar T75 flasks at 37° C. in a humidified atmosphere of 95% air-5% CO2. For experiments HepG2 cells were incubated in RPMI-1640 media without fetal calf serum. All experiments were carried out in 6 wells plates.

Reagents: Human IL-6 was obtained from Sigma Aldrich (Zwijndrecht, the Netherlands).

Incubations: HepG2 cells were grown in 6 wells plates to confluence, washed with DMEM (Dulbecco minimal essential medium) and subsequently incubated during 24-h with IL-6 and the custom media (RPMI-1640; RPMI-1640 select amino kit Gibco BRL). After the incubations, the media were collected and stored at −20° C. until analysis. The cells were then washed with phosphate-buffered saline (PBS) and lysed with 0.1M NaOH. The protein content was determined using the Bio-Rad Protein assay (Dye Reagent Concentrate, Bio-Rad Laboratories, Inc., Hercules Calif., USA) using bovine albumin (Sigma Aldrich) as standard. The data are presented as μg acute-phase protein in the supernatant per milligram protein cell content.

Antibodies: Rabbit anti-human fibrinogen and horseradish peroxidase (HRP)—conjugated rabbit anti-human fibrinogen were obtained from DakoCytomation (Denmark A/S, Glostrup, Denmark), anti-human albumin, HRP-conjugated anti-human albumin were obtained from Dade Behring B.V. (Leusden, the Netherlands).

Antigens: Human fibrinogen and human albumin were obtained from Sigma Aldrich (Zwijndrecht, the Netherlands).

ELISAs: Albumin and fibrinogen: 96-well flat-bottom Costar EIA/RIA plates were coated overnight with primary antibody in PBS at predetermined optimal concentration. After each incubation step, the plates were washed with 0.1% Tween-20 (Merck Eurolab B.V., Roden, the Netherlands) in PBS. After washing, the plates were blocked with 5% Protifar (Nutricia B.V., Zoetermeer, the Netherlands) in PBS during 90 min. Subsequently, the samples and antigen were incubated in 0.1% Tween-20 in PBS during 90 min. Next, the plates were incubated with HRP-conjugated antibodies in 0.1% Tween-20 in PBS during 90 min. The colorimetric reaction was carried out with undiluted 1-Step Ultra TMB-ELISA (Pierce, Rockland, Ill., USA). The reaction was stopped with 2 M sulphuric acid. The absorbances of the samples were measured at λ=450 nm.

Amino acid analysis: The amino acid concentrations in the media were determined with HPLC, using ortho-phtaldialdehyde as derivatisation reagent and L-norvaline as internal standard. The method was adapted from van Eijk et al.⁸

Statistics: To determine significant differences between values, multiple pair-wise comparisons were conducted with Student's t-test. P-values below 0.05 were interpreted as statistically significant.

### Results

Firstly, the in vitro HegG2 model was validated for the production of acute-phase proteins. Fibrinogen secretion (interpreted as synthesis) showed a concentration-dependent relationship with IL-6 (range 1-10 ng/ml).

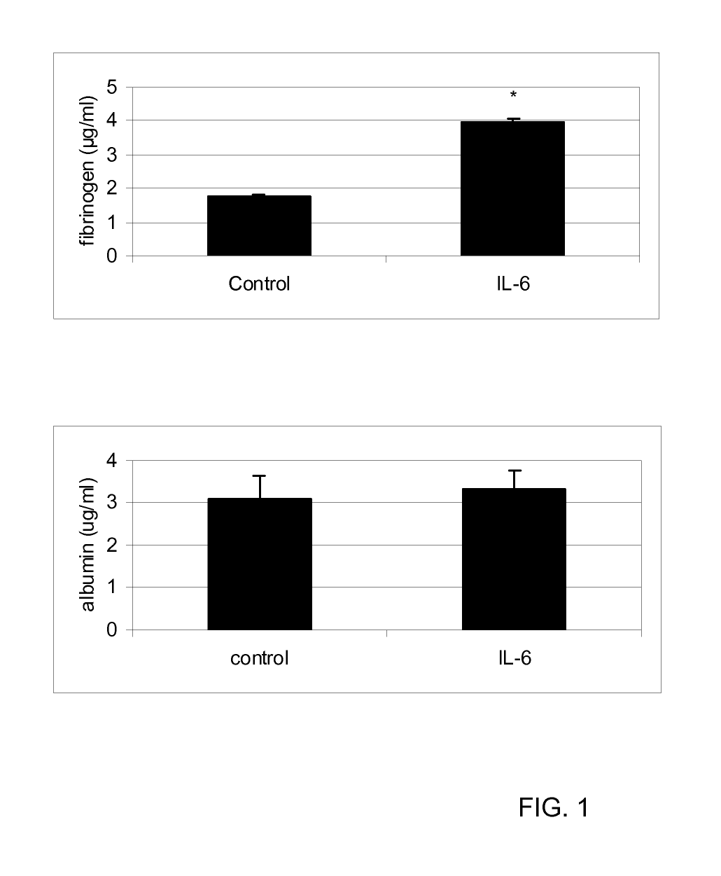

Albumin was secreted by the HepG2 cells at a rate of approximately 1.5 μg/ml per 24-h. There were no significant effects on albumin secretion observed after incubation with IL-6 (FIG. 1) or any of the other cytokines or hormones (and mixtures).

The effects of the cytokines IL-1β and TNFα, and the hormones insulin and dexamethasone on basal and IL-6 induced fibrinogen secretion are shown in FIG. 2. IL-1β inhibited the inducible effect of IL-6 on fibrinogen secretion by approximately 25% (P<0.001), both in the presence or absence of additional supplementation with TNFα. When added in combination with IL-6, insulin had an inhibitory effect (+50%) (P<0.01), and dexamethasone had an enhancing effect (+20%) on IL-6 induced fibrinogen secretion (P<0.001).

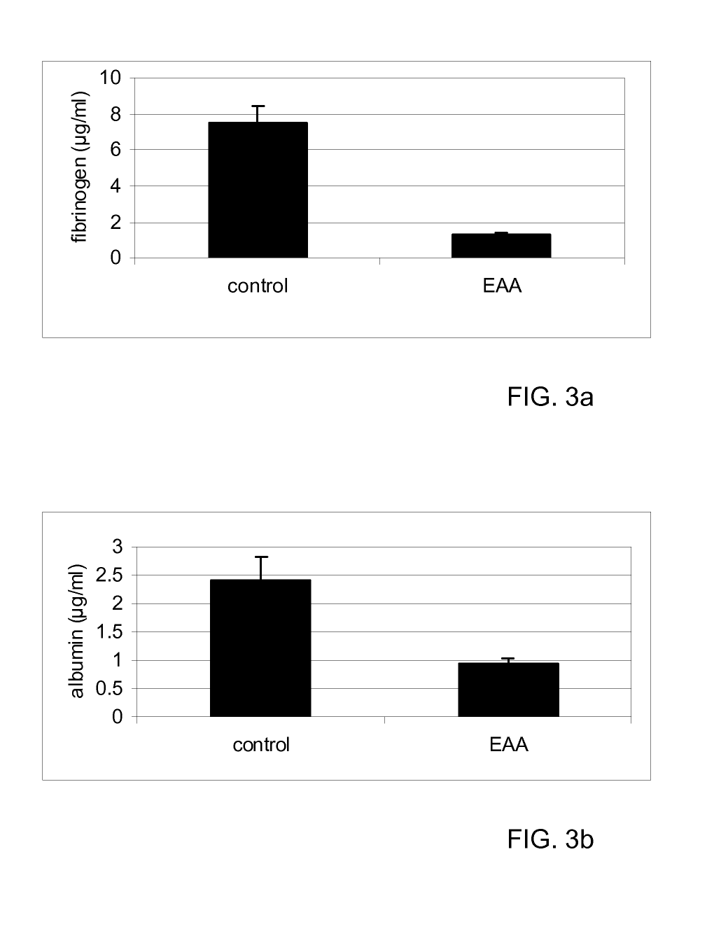

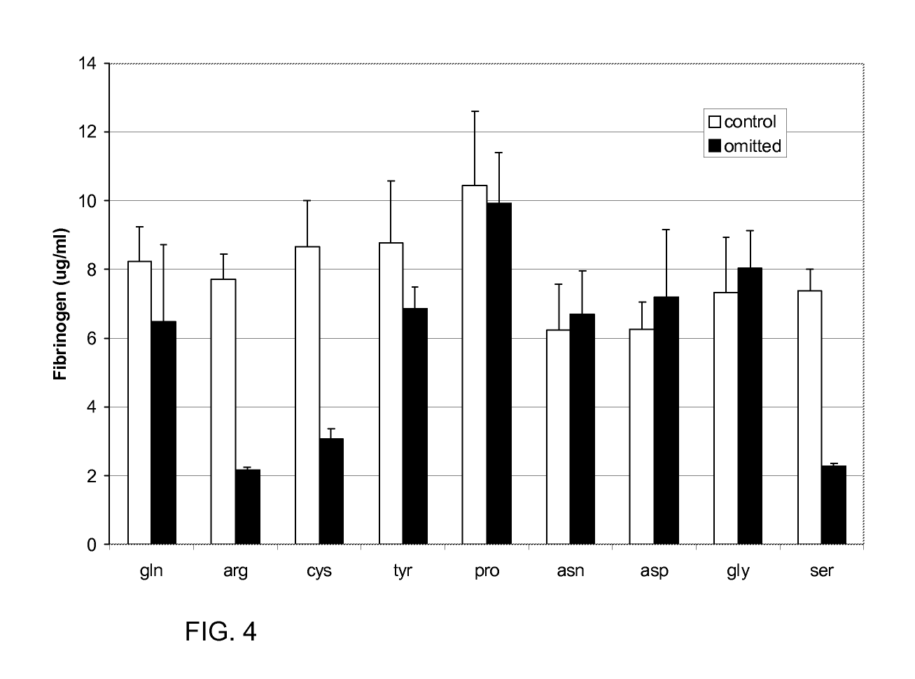

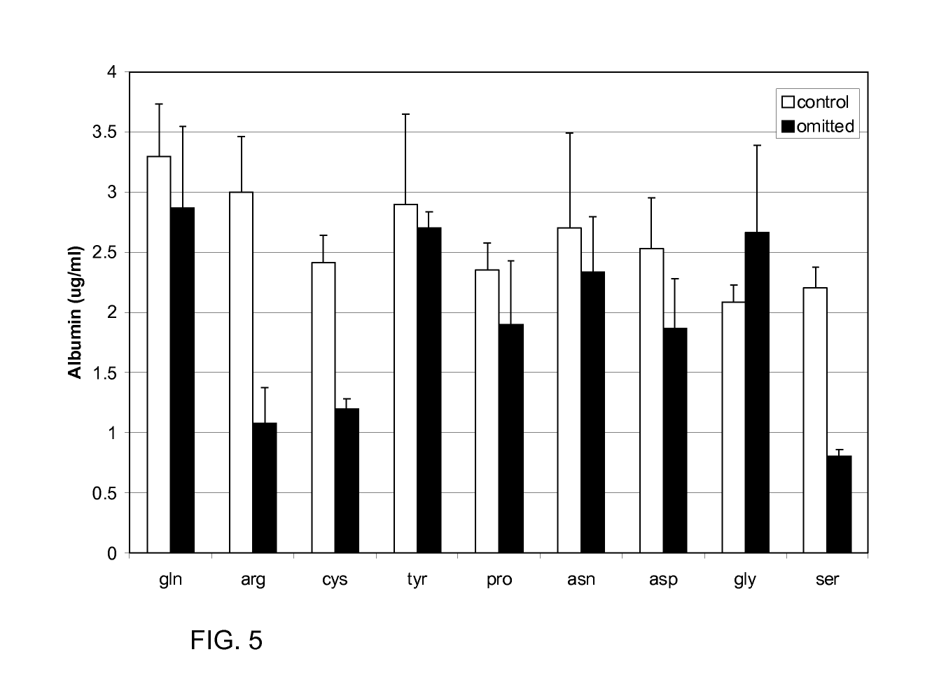

A medium with only essential amino acids present (isonitrogenously compensated) was prepared, to investigate whether essential amino acids were enough to mount an acute-phase protein response in the HepG2 cells. The results are shown in FIG. 3a and FIG. 3b. The secretion of both fibrinogen and albumin were significantly decreased in cell culture media with only essential amino acids. Subsequently, an experiment was carried out to investigate what other amino acids were relevant for acute-phase protein synthesis in the HepG2 cell line. Therefore, different media were prepared in which one nonessential amino acid was omitted and isonitrogenously compensated with the other amino acids. If arginine, cysteine or serine were omitted from the media, the secretion of both fibrinogen and albumin was significantly decreased (to levels not significantly different from media without amino acids) and designated as essential for acute-phase protein synthesis in HepG2 cells (FIG. 4 and FIG. 5). In contrast, if any of the other nonessential amino acids was omitted from the media, no significant effects were observed on acute-phase protein secretion of the HepG2 cells.

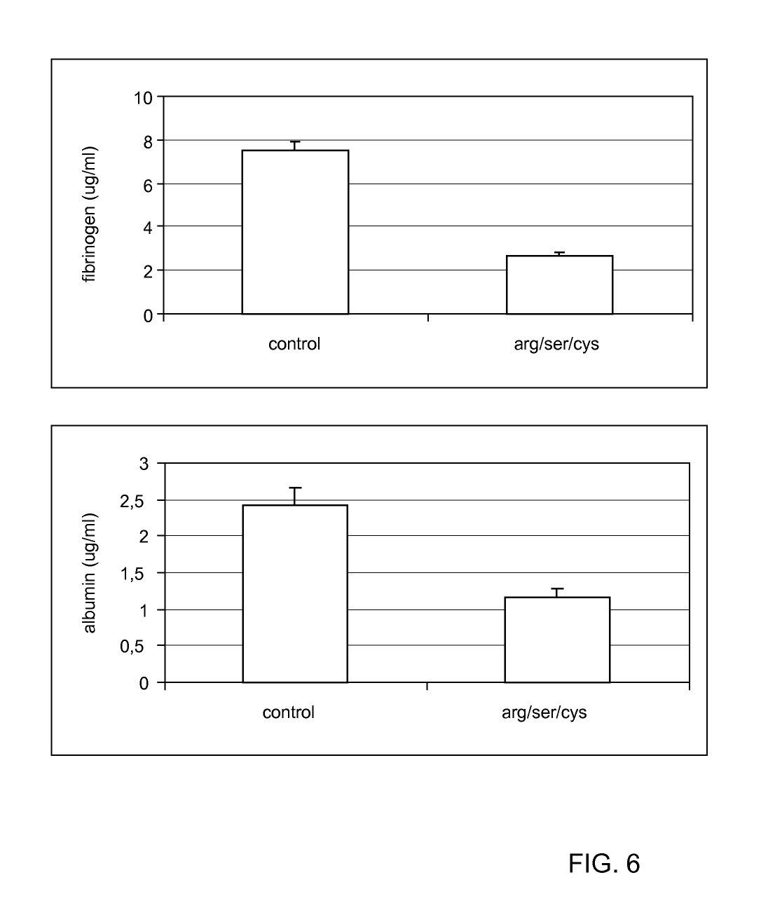

In an experiment carried out with all essential amino acids plus arginine, cysteine and serine still a significant reduction was observed in both albumin and fibrinogen (FIG. 6).

Therefore the amino acid consumption of the HepG2 cells after IL-6 stimulation in complete medium was measured. The results are shown in Table 2.

TABLE 2
Concentration (in μM) of amino acids in RPMI-
1640 media, after 0 and 24 hours from administration

| | T = 0 h | T = 24 h |
|---|---|---|
| Asp | 377 ± 18 | 339 ± 15 * |
| Glu | 408 ± 20 | 465 ± 23 * |
| Asn | 358 ± 17 | 331 ± 15 |
| Ser | 356 ± 14 | 344 ± 17 |
| His | 278 ± 7 | 260 ± 12 |
| Gln | 392 ± 14 | 212 ± 3 ** |
| Gly | 661 ± 40 | 607 ± 37 |
| Thr | 327 ± 17 | 294 ± 13 |
| Arg | 292 ± 11 | 267 ± 12 |
| Ala | 213 ± 5 | 144 ± 8 * |
| Tyr | 274 ± 12 | 246 ± 24 |
| Val | 383 ± 15 | 328 ± 13 * |
| Met | 362 ± 11 | 360 ± 16 |
| Trp | 253 ± 8 | 248 ± 11 |
| Phe | 326 ± 16 | 298 ± 13 |
| Ile | 426 ± 12 | 324 ± 11 * |
| Leu | 368 ± 8 | 277 ± 10 * |
| Lys | 202 ± 13 | 181 ± 11 |

n = 6.
* p < 0.05;
** p < 0.001.

A decrease in concentration of aspartate, glutamine, alanine, valine, leucine and isoleucine was observed and a significant increase of the glutamate concentration was observed. No significant differences in the media of other amino acids were observed. These data indicate a higher use of the branch chain amino acids (valine, leucine and isoleucine) from the essential amino acid pool and a higher use of aspartate, glutamine and alanine from the non-essential pool.

### Example 3

### Glycaemic Index determination

Definition

The glycaemic index (GI) of a carbohydrate provides a measure of its ability to raise postprandial glucose concentrations. High GI foods give higher postprandial blood glucose levels than those with a low GI. The GI of a carbohydrate also predicts the insulin response to that food.

The GI of a carbohydrate is calculated by assessing a 25 g two-hour glycaemic response with that of a subsequent 25 g carbohydrate standard glucose:

GI equals ‘Incremental area under blood glucose response curve for a test food containing 25 g of carbohydrate’ divided by ‘Corresponding area after equivalent carbohydrate portion of glucose’

Glycaemic Index Methodology

Available carbohydrate is defined for GI testing purposes as:

Total carbohydrate minus the indigestible carbohydrates (soluble and insoluble) that are from a physiological point dietary fibres (e.g. inulin, FOS, type 3 resistant starch)

The samples provided should be representative of the product as available to the consumer in the market place.

All foods submitted for testing are tested in vivo, that is, in 10 human subjects consuming amounts containing the equivalent of 25 g available carbohydrate. They are healthy subjects with no chronic diseases, diabetes or glucose impairment. Subjects have a BMI between 18.5-27 kg/m².

Reference food: The reference food is 25 g glucose powder dissolved in 250 mls water. Each person tests the reference food at least twice.

Test foods: The test foods are prepared according to manufacturer's instructions, representing the food as normally consumed. The test foods are consumed once only on separate occasions as a portion providing 25 g of available carbohydrate, defined as above.

Protocol Subjects: Subjects are tested in the morning after a 10-12 h overnight fast. Two fasting blood samples are taken (−5 & 0) 5 minutes apart after which subjects consume the test meal or reference food at an even rate over 15 minutes. Further blood samples are taken at 15, 30, 45, 60, 90 and 120 minutes after the beginning of the meal. The test meal and reference food should be consumed with a 250 mls drink of water. This remains constant for each of the tests in the series.

24 hrs prior to GI test: The day before each session, subjects refrain from drinking alcohol and avoid unusual levels of exercise and food intake. Subjects must have an evening meal based on a carbohydrate-rich food, such as rice, pasta, bread, potatoes and not too much fat. This meal should not include beans, pulses or legumes (to avoid a second meal effect the next morning). It is important that they eat dinner and not fast for more than 18 hours. Subjects are asked to be in a similar state each time they come in for a session. After they have eaten their evening meal, subjects fast for at least 10 hours overnight before the start of their test session the next morning. They can drink only water during the fasting period.

Blood sampling: Blood is obtained by finger pricking.

Blood is collected without clotting inhibitors (heparin, EDTA).

Glucose assay: Whole capillary blood or is measured by an automatic glucose analyzer. In this case, Hemocue glucose analysers are used.

Data analysis: The incremental area under the blood glucose response curve (iAUC), ignoring area beneath the baseline, is calculated geometrically as follows:

For times t0, t1, . . . to the blood glucose concentrations are G0, G1, . . . Gn, respectively:

$$iAUC = {\sum }_{n}^{x = 1} Ax$$

wherein Ax=the AUC for the xth time interval (i.e. between tx−1 and tx).

For the first time interval (i.e. x=1): if G1>G0, A1=(G1−G0)×(t1−t0)/2 otherwise, A1=0

For the other time intervals (i.e. x>1)

if Gx≧G0 and Gx−1≧G0, Ax={[(Gx−G0)/2]+(Gx−1−G0)/2}×(tx−tx−1)

if Gx>G0 and Gx−1<G0, Ax=[(Gx−G0)²/(Gx−Gx−1)]×(tx−tx−1)/2

if Gx≦G0 and Gx−1>G0, Ax=[(Gx−1−G0)²/(Gx−1−Gx)]×(tx−tx−1)/2

if Gx≦G0 and Gx−1≦G0, Ax=0

GI calculation: In individual subjects, the GI value is the iAUC for each food expressed as a percentage of the mean iAUC of the two reference foods (glucose). The GI of the test food is the mean GI±SEM of the 10 subjects.

Up to two outliers (an outlier is an individual whose GI differs from the mean by more than two SD) may be excluded from the data set. SEM should be within 20% of the mean.

### REFERENCES

1. Reeds P J, Fjeld C R, Jahoor F. Do the differences between the amino acid compositions of acute-phase and muscle proteins have a bearing on nitrogen loss in traumatic states? J Nutr. 1994;124(6):906-10.
2. Preston T, Slater C, McMillan D C, Falconer J S, Shenkin A, Fearon K C. Fibrinogen synthesis is elevated in fasting cancer patients with an acute-phase response. J Nutr 1998; 128(8):1355-60.
3. Jennings G, Bourgeois C, Elia M. The magnitude of the acute-phase protein response is attenuated by protein deficiency in rats. J Nutr 1992; 122(6):1325-31.
4. Curtis G E, McAtear C A, Formela L, Walsh A, Shenkin A. The effect of nutritional status on the cytokine and acute-phase protein responses to elective surgery. Cytokine 1995; 7(4):380-8.
5. Reid M, Badaloo A, Forrester T, Morlese J F, Heird W C, Jahoor F. The acute-phase protein response to infection in edematous and nonedematous protein-energy malnutrition. Am J Clin Nutr 2002; 76(6):1409-15. Available from http://www.ncbi.nlm.nih.gov/htbin-post/Entrez/query?db=m&for m=6&dopt=r&uid=12450910.
6. Fleck A. Clinical and nutritional aspects of changes in acute-phase proteins during inflammation. Proc Nutr Soc 1989; 48(3):347-54.
7. Reeds P J, Fjeld C R, Jahoor F. Do the differences between the amino acid compositions of acute-phase and muscle proteins have a bearing on nitrogen loss in traumatic states? J Nutr 1994; 124(6):906-10.
8. van Eijk H M, Rooyakkers D R, Deutz N E. Rapid routine determination of amino acids in plasma by high-performance liquid chromatography with a 2-3 microns Spherisorb ODS II column. J Chromatogr 1993; 620(1):143-8.

1. A combination of (i) serine, (ii) cysteine, (iii) arginine and (iv) at least one branched amino acid, for use in the therapeutic or prophylactic treatment of inflammation or infection, wherein the combination is to be administered enterally and wherein the combination forms part of a nutritional composition that has a glycaemic index below 55.

2. A combination of (i) serine, (ii) cysteine, (iii) arginine and (iv) at least one branched amino acid, for use in the therapeutic or prophylactic treatment of an imbalance in the metabolic use of amino acid resources from the body or from nutrition in a subject having an inflammation or an infection, wherein the combination is to be administered enterally and wherein the combination forms part of a nutritional composition that has a glycaemic index below 55.

3. A combination of (i) serine, (ii) cysteine, (iii) arginine and (iv) at least one branched amino acid, for use according to claim 2, wherein the therapeutic or prophylactic treatment of is a treatment of an imbalance in the metabolic use of amino acid resources from the body or from nutrition for acute-phase protein synthesis.

4. A combination for use in a treatment according to claim 1, wherein the combination forms part of a nutritional composition that has a glycaemic index below 55.

5. A combination for use in a treatment according to claim 1, further comprising (v) at least one component selected from the group consisting of phenylalanine, threonine, tryptophan, methionine and lysine.

6. A combination for use in a treatment according to claim 5, wherein the total relative amount of branched amino acids as a weight percentage of the total amount of leucine, isoleucine, valine, phenylalanine, threonine, tryptophan, methionine and lysine is in the range of 45-75 weight %.

7. A combination for use in a treatment according to claim 1, comprising a nutritional anti-inflammatory component.

8. A combination for use in a treatment according to claim 7, wherein the nutritional anti-inflammatory component is selected from the group consisting of eicosapentaenoic acid (EPA), docosahexaenoic acid (DHA), eicosatetraenoic acid (ESA), docosapentaenoic acid (DPA), glycine, and nutritional components inhibiting COX II activity.

9. A combination for use in a treatment according to claim 1, wherein the total relative amount of leucine, isoleucine, valine, phenylalanine, threonine, tryptophan, methionine and lysine, relative to the total amount of amino acids in the composition is in the range of 40-80 weight %.

10. A combination for use in a treatment according to claim 1, wherein the total relative amount of serine, cysteine and arginine, relative to the total amount of essential amino acids in the composition is in the range of 10-40 weight %.

11. A combination for use in a treatment according to claim 1, wherein said amino acid or said amino acids are provided in at least one of the following forms: as a free amino acid (including its zwitter ionic state); as a salt of free amino acid; as a peptide comprising one or more of the amino acid units; as a protein comprising one or more of the amino acid units, which protein may be intact or partially hydrolysed; cysteine as cystine; as a conjugate of the amino acid with a conjugating compound other than an amino acid, a protein, or a peptide, which conjugate is capable of being split into the free amino acid (or salt thereof), preferably in the gut or stomach or after absorption in the enterocytes or liver.

12. A combination for use in a treatment according to claim 1, wherein the combination comprises one or more peptides having 8 or more amino acid units, and the total content of said peptides is at least 50 weight %, based on total proteinaceous matter.

13. A combination for use in a treatment according to claim 1, wherein the inflammation is caused by a chronic infection, a chronic disease, a systemic elevation of metabolites causing inflammation or by increased concentrations of auto-immune complexes.

14. A combination for use in a treatment according to claim 1, wherein the concentration of an acute phase protein in blood of the subject to be treated is more than 25% above a maximum normal physiological value in healthy individuals or more than 25% below a minimum normal physiological value in healthy individuals.

15. A combination for use in a treatment according to claim 3, wherein the acute phase protein is selected from the group consisting of haptoglobulin, serum amyloid protein, mannose binding protein, ferritin, ceruloplasmin, serpin, tyransthyretin, transferrin, fibrinogen, pre-albumin and transcortin.

16. A combination for use in a treatment according to claim 1, wherein the subject to be treated is a cancer patient, an HIV-infected subject, an AIDS patient, a COPD patient, a subject with renal failure, a subject with heart failure, a subject with insulin resistance, a subject having rheumatoid arthritis or osteoarthritis, a subject having pancreatitis, a subject having hepatitis, a subject having inflammatory bowel disease, a subject having Crohn's disease, a subject having an organ graft, a subject recovering from lesions applied to a body during surgery, a critically-ill subject, a terminally ill subject, an intoxicated individual or a subject suffering from an allergy.

17. (canceled)

18. Combination for use according to claim 1, wherein the combination is to be administered orally.

19. Nutritional composition comprising 4-9 weight % serine, 0.7-8 weight % of cysteine, 2-4 weight % arginine 8.0-20 weight % leucine, 3-8.0 weight % isoleucine, 3-8.0 weight % valine, 2.5-8.0 weight % phenylalanine, 3-7 weight % threonine, 1.0-3 weight % trypthophan, 1.0-3.0 weight % methionine, 7.5-12 weight % lysine, and 0-10 weight % glycine, all based on total weight of proteinaceous matter, and wherein the nutritional composition has a glycaemic index below 55.

20. Nutritional composition according to claim 19, comprising at least one additional ingredient selected from the group consisting of digestible carbohydrates, indigestible carbohydrates and other dietary fibres, trace elements, minerals, vitamins, proteinaceous matter other than proteinaceous matter providing said amino acids, lipids, and inhibitors of COX II activity.

21. Nutritional composition according to claim 20, having a glycaemic index of 5-45.

22. Nutritional composition according to claim 20, comprising one or more ω-3 polyunsaturated fatty acid(s), the total amount of ω-3 polyunsaturated fatty acid(s), based on total proteinaceous matter, being at least 4 weight %.

23. The nutritional composition of claim 19 being an enteral food composition.

24. The combination of claim 5 including phenylalanine, threonine, tryptophan, methionine and lysine.
